(19) United States
(12) Patent Application Publication
(10) Pub. No.: US 2012/0197163 A1
(43) Pub. Date: Aug. 2, 2012

(54) PATTERNED CONTROL OF ULTRASOUND FOR NEUROMODULATION

(76) Inventor: David J. Mishelevich, Playa del Rey, CA (US)

(21) Appl. No.: 13/360,600

(22) Filed: Jan. 27, 2012

Related U.S. Application Data
(60) Provisional application No. 61/436,607, filed on Jan. 27, 2011.

(51) Int. Cl. A61N 7/00

(52) U.S. Cl. 601/2

(57) ABSTRACT

Disclosed are methods and devices for ultrasound-mediated non-invasive deep brain neuromodulation impacting one or a plurality of points in a neural circuit using patterned inputs. These are applicable whether the ultrasound beams intersect at the targets or not. Depending on the application, this can produce short-term effects (as in the treatment of post-surgical pain) or long-term effects in terms of Long-Term Potentiation (LTP) or Long-Term Depression (LTD) to treat indications such as neurologic and psychiatric conditions. The ultrasound transducers are used with control of frequency, firing pattern, and intensity to produce up-regulation or down-regulation.

### CROSS REFERENCE TO RELATED APPLICATIONS

This patent application claims priority to Provisional Patent Application Number 61/436,607, filed Jan. 22, 2011, entitled “PATTERNED CONTROL OF ULTRASOUND FOR NEUROMODULATION.” The disclosures of this patent application are herein incorporated by reference in their entirety.

### INCORPORATION BY REFERENCE

All publications, including patents and patent applications, mentioned in this specification are herein incorporated by reference in their entirety to the same extent as if each individual publication was specifically and individually cited to be incorporated by reference.

### FIELD OF THE INVENTION

Described herein are systems and methods for Ultrasound Neuromodulation including one or a plurality of ultrasound sources for stimulation of target deep brain regions to up-regulate or down-regulate neural activity.

### BACKGROUND OF THE INVENTION

It has been demonstrated that focused ultrasound directed at neural structures can stimulate those structures. If neural activity is increased or excited, the neural structure is said to be up-regulated; if neural activated is decreased or inhibited, the neural structure is said to be down-regulated. Down regulation means that the firing rate of the neural target has its firing rate decreased and thus is inhibited and up regulation means that the firing rate of the neural target has its firing rate increased and thus is excited. Neural structures are usually assembled in circuits. For example, nuclei and tracts connecting them make up a circuit. The potential application of ultrasonic therapy of deep-brain structures has been suggested previously (Gavrilov L R, Tsirulnikov E M, and I A Davies, “Application of focused ultrasound for the stimulation of neural structures,” Ultrasound Med Biol. 1996; 22(2): 179-92. and S. J. Norton, “Can ultrasound be used to stimulate nerve tissue?,” BioMedical Engineering OnLine 2003, 2: 6). Norton notes that while Transcranial Magnetic Stimulation (TMS) can be applied within the head with greater intensity, the gradients developed with ultrasound are comparable to those with TMS. It was also noted that monophasic ultrasound pulses are more effective than biphasic ones. Instead of using ultrasonic stimulation alone, Norton applied a strong DC magnetic field as well and describes the mechanism as that given that the tissue to be stimulated is conductive that particle motion induced by an ultrasonic wave will induce an electric current density generated by Lorentz forces.

The effect of ultrasound is at least two fold. First, increasing temperature will increase neural activity. An increase up to 42 degrees C. (say in the range of 39 to 42 degrees C.) locally for short time periods will increase neural activity in a way that one can do so repeatedly and be safe. One needs to make sure that the temperature does not rise about 50 degrees C. or tissue will be destroyed (e.g., 56 degrees C. for one second). This is the objective of another use of therapeutic application of ultrasound, ablation, to permanently destroy tissue (e.g., for the treatment of cancer). An example is the ExAblate device from InSightec in Haifa, Israel. The second mechanism is mechanical perturbation. An explanation for this has been provided by Tyler et al. from Arizona State University (Tyler, W. J., Y. Tufail, M. Finsterwald, M. L. Tauchmann, E. J. Olsen, C. Majestic, “Remote excitation of neuronal circuits using low-intensity, low-frequency ultrasound,” PLoS One 3(10): e3511, doi:10.137/1/journal.pone.0003511, 2008)) where voltage gating of sodium channels in neural membranes was demonstrated. Pulsed ultrasound was found to cause mechanical opening of the sodium channels that resulted in the generation of action potentials. Their stimulation is described at Low Intensity Low Frequency Ultrasound (LILFU). They used bursts of ultrasound at frequencies between 0.44 and 0.67 MHz, lower than the frequencies used in imaging. Their device delivered 23 milliwatts per square centimeter of brain—a fraction of the roughly 180 mW/cm² upper limit established by the U.S. Food and Drug Administration (FDA) for womb-scanning sonograms; thus such devices should be safe to use on patients. Ultrasound impact to open calcium channels has also been suggested.

Alternative mechanisms for the effects of ultrasound may be discovered as well. In fact, multiple mechanisms may come into play, but, in any case, this would not effect this invention.

Approaches to date of delivering focused ultrasound vary. Bystritsky (U.S. Pat. No. 7,283,861, Oct. 16, 2007) provides for focused ultrasound pulses (FUP) produced by multiple ultrasound transducers (said preferably to number in the range of 300 to 1000) arranged in a cap place over the skull to produce a multi-beam output. These transducers are coordinated by a computer and used in conjunction with an imaging system, preferable an fMRI (functional Magnetic Resonance Imaging), but possibly a PET (Positron Emission Tomography) or V-EEG (Video-Electroencephalography) device. The user interacts with the computer to direct the FUP to the desired point in the brain, sees where the stimulation actually occurred by viewing the imaging result, and thus adjusts the position of the FUP according. The position of focus is obtained by adjusting the phases and amplitudes of the ultrasound transducers (Clement and Hynynen, “A non-invasive method for focusing ultrasound through the human skull,” Phys. Med. Biol. 47 (2002) 1219-1236). The imaging also illustrates the functional connectivity of the target and surrounding neural structures. The focus is described as two or more centimeters deep and 0.5 to 1000 mm in diameter or preferably in the range of 2-12 cm deep and 0.5-2 mm in diameter. Either a single FUP or multiple FUPs are described as being able to be applied to either one or multiple live neuronal circuits. It is noted that differences in FUP phase, frequency, and amplitude produce different neural effects. Low frequencies (defined as below 300 Hz.) are inhibitory. High frequencies (defined as being in the range of 500 Hz to 5MHz are excitatory and activate neural circuits. This works whether the target is gray or white matter. Repeated sessions result in long-term effects. The cap and transducers to be employed are preferably made of non-ferrous material to reduce image distortion in fMRI imaging. It was noted that if after treatment the reactivity as judged with fMRI of the patient with a given condition becomes more like that of a normal patient, this may be indicative of treatment effectiveness. The FUP is to be applied 1 ms to 1 s before or after the imaging. In addition a CT (Computed Tomography) scan can be run to gauge the bone density and structure of the skull.

Deisseroth and Schneider describe an alternative approach (U.S. patent application Ser. No. 12/263,026 published as US 2009/0112133 A1, Apr. 30, 2009) in which modification of neural transmission patterns between neural structures and/or regions is described using sound (including use of a curved transducer and a lens) or RF. The impact of Long-Term Potentiation (LTP) and Long-Term Depression (LTD) for durable effects is emphasized. It is noted that sound produces stimulation by both thermal and mechanical impacts. The use of ionizing radiation also appears in the claims.

Adequate penetration of ultrasound through the skull has been demonstrated (Hynynen, K. and F A Jolesz, “Demonstration of potential noninvasive ultrasound brain therapy through an intact skull,” Ultrasound Med Biol, 1998 February; 24(2): 275-83 and Clement G T, Hynynen K (2002) A non-invasive method for focusing ultrasound through the human skull. Phys Med Biol 47: 1219-1236.) . Ultrasound can be focused to 0.5 to 2 mm as TMS to 1 cm at best.

The targeting can be done with one or more of known external landmarks, an atlas-based approach (e.g., Tailarach or other atlas used in neurosurgery) or imaging (e.g., fMRI or Positron Emission Tomography). The imaging can be done as a one-time set-up or at each session although not using imaging or using it sparingly is a benefit, both functionally and the cost of administering the therapy, over Bystritsky (U.S. Pat. No. 7,283,861) which teaches consistent concurrent imaging.

While ultrasound can be focused down to a diameter on the order of one to a few millimeters (depending on the frequency), whether such a tight focus is required depends on the conformation of the neural target. For example, some targets, like the Cingulate Gyms, are elongated and will be more effectively served with an elongated ultrasound field at the target.

It would be preferable to not only stimulate single or multiple targets synchronously, but to have patterns applied both to a single ultrasound transducer and to the stimulation relationships among multiple such transducers.

### SUMMARY OF THE INVENTION

It is the purpose of this invention to provide an ultrasound device delivering enhanced non-invasive superficial or deep-brain neuromodulation using pulse patterns impacting one or a plurality of points in a neural circuit to produce acute effects or Long-Term Potentiation (LTP) or Long-Term Depression (LTD) using up-regulation or down-regulation. Multiple points in a neural circuit can all up regulated, all down regulated or there can be a mixture. Typically LTP is obtained by up-regulation obtained through neuromodulation and LTD obtained by down-regulation obtained through neuromodulation. Two different targets may have different optimal frequency stimulations (even if both up-regulated and down-regulated).

In this invention, this is achieved by individually controlling the pulse pattern applied to each of the ultrasound transducers generating ultrasound beams impacting individual targets. The pulse patterns can be applied to individual ultrasound transducers hitting individual targets or sets of transducers applying ultrasound neuromodulation on a given target using non-intersecting or intersecting ultrasound beams. Pulse patterns can vary in one or both of timing or intensity. Timing patterns may vary either in frequency or inter-pulse or inter-train intervals (e.g., one pulse followed by two pulses with a shorter inter-pulse interval and repeat) for each individual ultrasound transducer.

To assess the efficacy of the patterned neuromodulation, ancillary monitoring or imaging may be employed.

### BRIEF DESCRIPTION OF THE DRAWINGS

FIG. 1: Table of neuromodulation patterns.

FIG. 2: Block diagram of neural circuit in the brain for addiction.

FIG. 3: Four ultrasound transducers targeting four targets in the neural addiction circuit including the Orbito-Frontal Cortex (OFC), the Dorsal Anterior Cingulate Gyms (DACG), the Insula, and the Nucleus Accumbens.

FIG. 4: Neural circuit allowing alternative effects depending on whether the circuit is up regulated or down regulated.

FIG. 5: Block diagram of the mechanism for controlling the multiple ultrasound beams.

### DETAILED DESCRIPTION OF THE INVENTION

The invention is an ultrasound device using non-intersecting beams or intersecting beams delivering enhanced non-invasive deep brain or superficial deep-brain neuromodulation using patterned stimulation impacting one or a plurality of points in a neural circuit providing for up-regulation or down-regulation of neural targets, as applicable, to produce acute effects (as in the treatment of post-surgical pain) or Long-Term Potentiation (LTP) or Long-Term Depression (LTD). Patterns can be applied to multiple beams that intersect to stimulate a single target. One reason for using such intersecting beams is to divide the applied power into multiple components so that the power can be utilized to adequately neuromodulate the intended target without over-stimulating the tissues between the ultrasound transducers and the target and causing undesirable side effects such as seizures.

The stimulation frequency for inhibition is 300 Hz or lower (depending on condition and patient). The stimulation frequency for excitation is in the range of 500 Hz to 5 MHz. In this invention, the ultrasound acoustic frequency is in range of 0.3 MHz to 0.8 MHz to permit effective transmission through the skull with power generally applied less than 180 mW/cm² but also at higher target- or patient-specific levels at which no tissue damage is caused. The acoustic frequency (e.g., 0.44 MHz that permits the ultrasound to effectively penetrate through skull and into the brain) is gated at the lower rate to impact the neuronal structures as desired (e.g., say 300 Hz for inhibition (down-regulation) or 1 kHz for excitation (up-regulation). If there is a reciprocal relationship between two neural structures (i.e., if the firing rate of one goes up the firing rate of the other will decrease), it is possible that it would be appropriate to hit the target that is easiest to obtain the desired result. For example, one of the targets may have critical structures close to it so if it is a target that would be down regulated to achieve the desired effect, it may be preferable to up-regulate its reciprocal more-easily-accessed or safer reciprocal target instead. The frequency range allows penetration through the skull balanced with good neural-tissue absorption. Ultrasound therapy can be combined with therapy using other devices (e.g., Transcranial Magnetic Stimulation (TMS), transcranial Direct Current Stimulation (tDCS), and/or Deep Brain Stimulation (DBS) using implanted electrodes).

The lower bound of the size of the spot at the point of focus will depend on the ultrasonic frequency, the higher the frequency, the smaller the spot. Ultrasound-based neuromodulation operates preferentially at low frequencies relative to say imaging applications so there is less resolution. As an example, let us have a hemispheric transducer with a diameter of 3.8 cm. At a depth approximately 7 cm the size of the focused spot will be approximately 4 mm at 500 kHz where at 1 Mhz, the value would be 2 mm. Thus in the range of 0.4 MHz to 0.7 MHz, for this transducer, the spot sizes will be on the order of 5 mm at the low frequency and 2.8 mm at the high frequency.

Transducer array assemblies of the type used in this invention may be supplied to custom specifications by Imasonic in France (e.g., large 2D High Intensity Focused Ultrasound (HIFU) hemispheric array transducer)(Fleury G., Berriet, R., Le Baron, O., and B. Huguenin, “New piezocomposite transducers for therapeutic ultrasound,” 2^{nd} International Symposium on Therapeutic Ultrasound—Seattle—31/07—Feb. 8, 2002), typically with numbers of sound transducers of 300 or more. Blatek and Keramos-Etalon in the U.S. are other custom-transducer suppliers. The power applied will determine whether the ultrasound is high intensity or low intensity (or medium intensity) and because the sound transducers are custom, any mechanical or electrical changes can be made, if and as required.

The locations and orientations of the transducers and their stimulation patterns in this invention can be calculated by locating the applicable targets relative to atlases of brain structure such as the Tailarach atlas or established though fMRI, PET, or other imaging of the head of a specific patient. Using multiple ultrasound transducers two or more targets can be targeted simultaneously or sequentially. The ultrasonic firing patterns can be tailored to the response type of a target or the various targets hit within a given neural circuit.

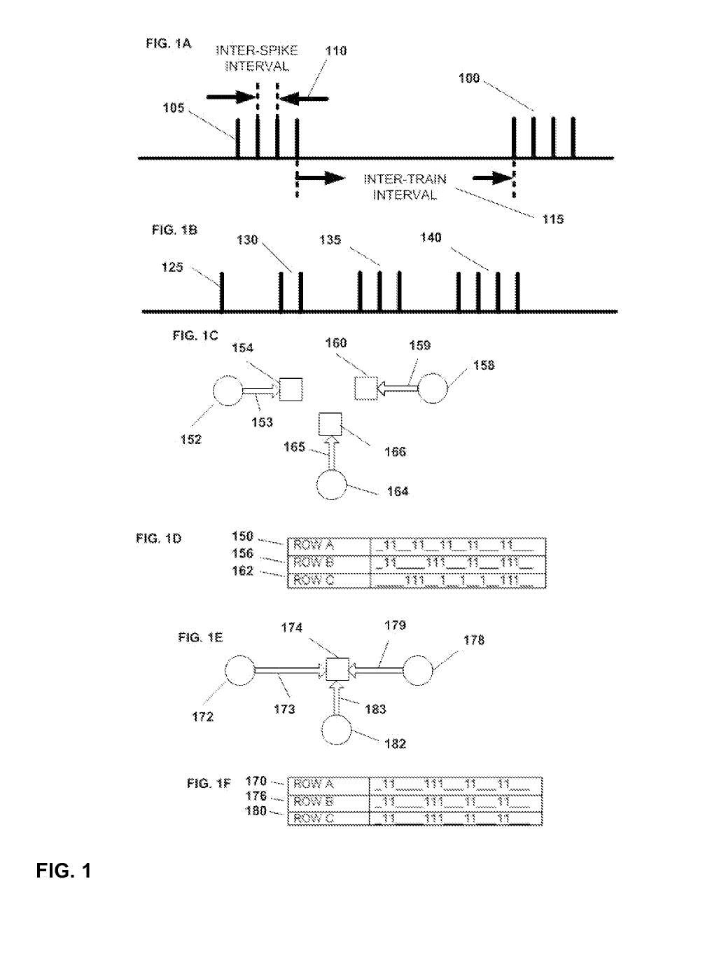

FIG. 1 illustrates examples of patterns. In FIG. 1A, Pulse trains 100 are composed of one or a plurality of sets of pulses (e.g., singletons, pairs, triplets, etc.) made up of individual pulses 105 with inter-spike intervals 110 with the trains separated by inter-pulse-train intervals 115. If the set of inter-pulse intervals 130 is of length zero, then the train is continuous. FIG. 1B illustrates examples of an individual pulse singlet 125 as well as pulse sets pulse pair 130, pulse triplet 135, and pulse quadruplet 140. The elements of a train may the same or they may vary. For example, a pair of pulses may alternate with a triplet of pulses and/or the inter-pulse-train intervals may vary. Patterns applied may be either fixed or random. Sample patterns include pairs, triplets, or other multiplicates, theta burst stimulations, alternating simple patterns (e.g., alternating pairs with triplets), changing frequencies during stimulations (e.g., for a singlet ramping up the stimulation frequency from 5 Hz. to 20 Hz. over a period of 15 stimulations and then ramping down the stimulation from 20 Hz to 5 Hz. in the next 15 stimulations where the frequencies increase and decrease can be linear or non-linear), and others. Variable or fixed patterns can apply to individual targets or among targets. An example of another pattern is Theta-Burst Stimulation (TBS) that consists of short bursts (e.g., 3) of high-frequency pulses impulses repeated at 5 Hz (the frequency of the theta rhythm in the EEG). In some cases the pattern applied to a given neural target or neural circuit may constitute a natural rhythm for that target or circuit and may even include resonance. Patterns include variations in rate or intensity. The relationship between the applied frequency, timing pattern and applied intensity pattern can be independently varied, dependently varied, independently fixed, and dependently fixed.

FIG. 1C shows a diagram of three ultrasound transducers 152, 158, and 164 with respective ultrasound beams 153, 159, and 165 impacting three targets 154, 160, and 166 supporting patterned stimulation where multiple ultrasonic transducers are each aimed at different targets. Depending on the characteristics of the targets, the stimulation patterns of each transducer in a set of transducers may be the same or different. FIG. 1D illustrates examples of stimulation patterns for the case shown in FIG. 1C. Stimulation-pattern row 150 shows the stimulation pattern for ultrasound transducer 152 aimed at target 154. Stimulation-pattern row 156 shows the stimulation pattern for ultrasound transducer 158 aimed at target 160. Stimulation-pattern row 162 shows the stimulation pattern for ultrasound transducer 164 aimed at target 166.

FIG. 1E shows a diagram of three ultrasound transducers 172, 178, and 182 with respective ultrasound beams 173, 179, 183 impacting common target 174 supporting patterned stimulation where multiple ultrasonic transducers are each aimed at the same target. FIG. 1F illustrates examples of stimulation patterns for the case shown in FIG. 1E. Stimulation-pattern row 170 shows the stimulation pattern for ultrasound transducer 172 aimed at target 174. Stimulation-pattern row 176 shows the stimulation pattern for ultrasound transducer 178 also aimed at target 174. Stimulation-pattern row 180 shows the stimulation pattern for ultrasound transducer 182 again also aimed at target 174. Even when a common target is neuromodulated, adjustment of stimulation parameters may moderate or eliminate a problem with side effects from the neuromodulation.

In the case of synchronous patterns, the same pattern is applied to multiple targets. In the case of asynchronous patterns, different patterns are applied to different targets. In the case of independent patterns when two different patterns are applied to different targets, when one pattern is changed, the other is not changed or not in changed in the same way. If one or a plurality of targets are all up-regulated or all down-regulated or there is a mixture of such regulation, different frequencies can be used to optimize the desired effects on the various targets (e.g., one up-regulation done at 5 Hz. and another at 10 Hz.). Invention includes the concept of having different patterns for each of a pair of bilateral structures. For example, in the treatment of addiction, neuromodulating the Insula involves down regulating the Insula on the right side.

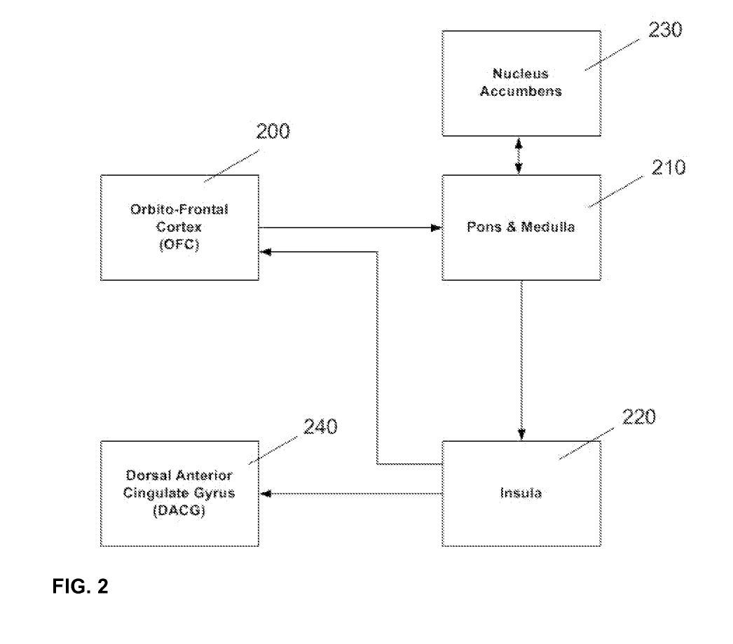

FIG. 2 shows a set of important targets for the treatment of addiction. Five targets are shown, Orbito-Frontal Cortex (OFC) 200, Pons & Medulla 210, Insula 220, Nucleus Accumbens 230, and Dorsal Anterior Cingulate Gyms (DACG) 240.

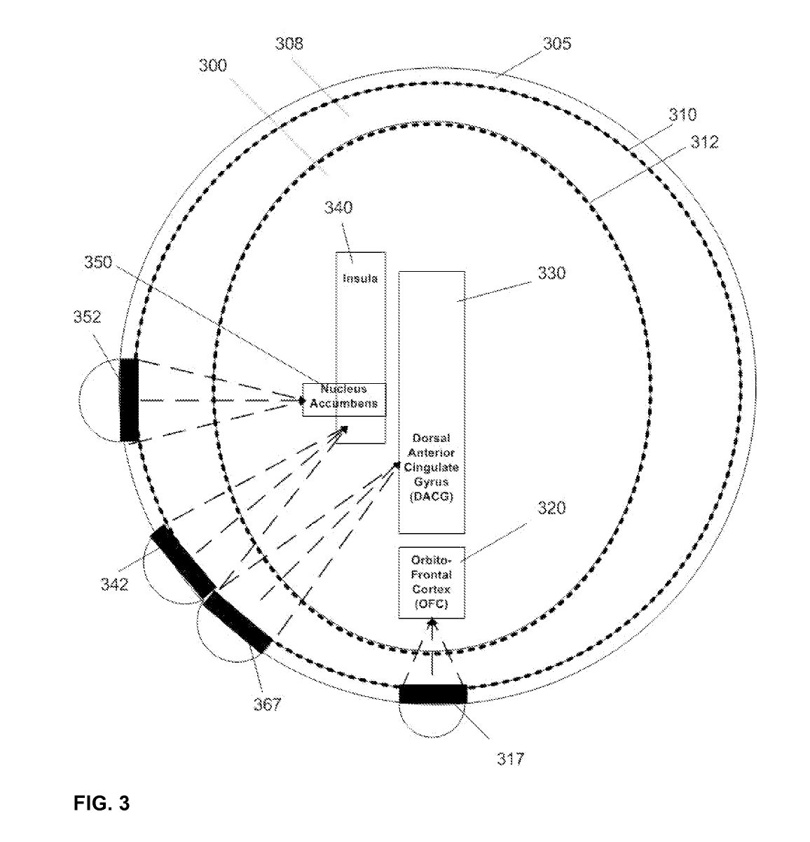

FIG. 3 illustrates within head 300 four targets related to the treatment of addiction from FIG. 2, Orbito-Frontal Cortex (OFC) 320, Dorsal Anterior Cingulate Gyms (DACG) 330, Insula 340, and Nucleus Accumbens 350. Mounted on frame 305 are ultrasound transducers 317 targeting OFC 320, 367 targeting DACG 330, 342 targeting Insula 340, and 352 targeting Nucleus Accumbens 350. Ultrasound transducers 317, 367, 342, and 352 have focused, non-intersecting ultrasound beams. To obtain effective transmission, each of the ultrasound beams is directed through ultrasound conduction medium 308 with layers of ultrasound conduction gel 310 between the ultrasound transducers lens faces and ultrasound conduction gel 312 between the ultrasound conduction medium 308 and that medium and the head 300. Examples of ultrasound conduction media include Dermasol from California Medical Innovations and silicone oil in a containment pouch. In an alternative embodiment instead of a band of ultrasound conduction medium being placed around the head, individual ultrasound conduction media are placed for each ultrasound transducers, again including ultrasound conduction gel layers between the transducer lens face and the conduction medium and also between the ultrasound conduction medium and the head. Pulsed patterns are then used to excite each transducer. To treat addiction, for the four targets being neuromodulated, the Orbito-Frontal Cortex (OFC) and the Nucleus Accumbens are up regulated and the Dorsal Anterior Cingulate Gyms (DACG) and the Insula are down regulated.

One or more targets can be targeted simultaneously or sequentially. Down regulation means that the firing rate of the neural target has its firing rate decreased and thus is inhibited and up regulation means that the firing rate of the neural target has its firing rate increased and thus is excited. The ultrasonic firing/timing patterns can be tailored to the response type of a target or the various targets hit within a given neural circuit.

In another embodiment the ultrasound beams intersect at the targets. This can be useful where one wants to increase the intensity level at a given target, but decrease the intensity of tissue intermediate between the output interface of the ultrasound transducer and the given target. In this invention, two or more beams intersect at a given target with appropriate patterns applied to each of the beams. Use of patterns and/or intersecting ultrasound beams avoids excessive stimulation of nearby structures that need to be protected.

In another embodiment, the neuromodulation of one or a plurality of ultrasound transducers is combined with the neuromodulation from one or a plurality of Transcranial Magnetic Stimulation (TMS) electromagnetic coils. In another embodiment, a viewing hole can be placed in an ultrasound transducer to provide an imaging port. Blatek, Imasonic and Keramos-Etalon can supply such configurations. In another embodiment auditory input can be a neuromodulation modality combined with ultrasound neuromodulation or ultrasound neuromodulation and Transcranial Magnetic Stimulation.

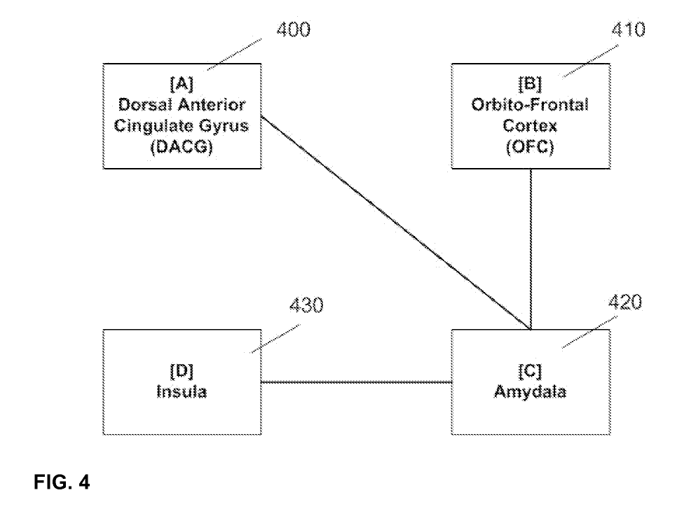

FIG. 4 illustrates the neural circuit representing the case where alternative effects can occur depending on whether the elements of the circuit are either up regulated or down regulated. Note in some cases in a given circuit not all the elements will be all up regulated or down regulated. In FIG. 4, blocks [A] 400, [B] 410, [C] 420, and [D] 430 represent neural elements that can be up regulated or down regulated. In this example, for one clinical effect, all are regulated in the direction to achieve that effect, and for the opposite clinical effect, all are regulated in the opposite direction. As a specific embodiment, for bipolar disorder, [A] 400 represents the Dorsal Anterior Cingulate Gyms (DACG), [B] 410 represents the Orbital-Frontal Cortex (OFC), [C] 420 represents the Amygdala, and [D] 430 represents the Insula. For the condition Bipolar Disorder, if the depressive phase is being treated, the OFC 410, the Amygdala 420, and left-located Insula 430 are down regulated, and the DACG 400 and right-located Insula are up regulated. On the other hand, if the manic phase is being treated, the OFC 410, the Amygdala 420, and left-located Insula 430 are up regulated, and the DACG 400 and right-located Insula 430 are down regulated. In a sense, the circuit is sped up or advanced to treat the depressive phase and slowed down or retarded to treat the manic phase.

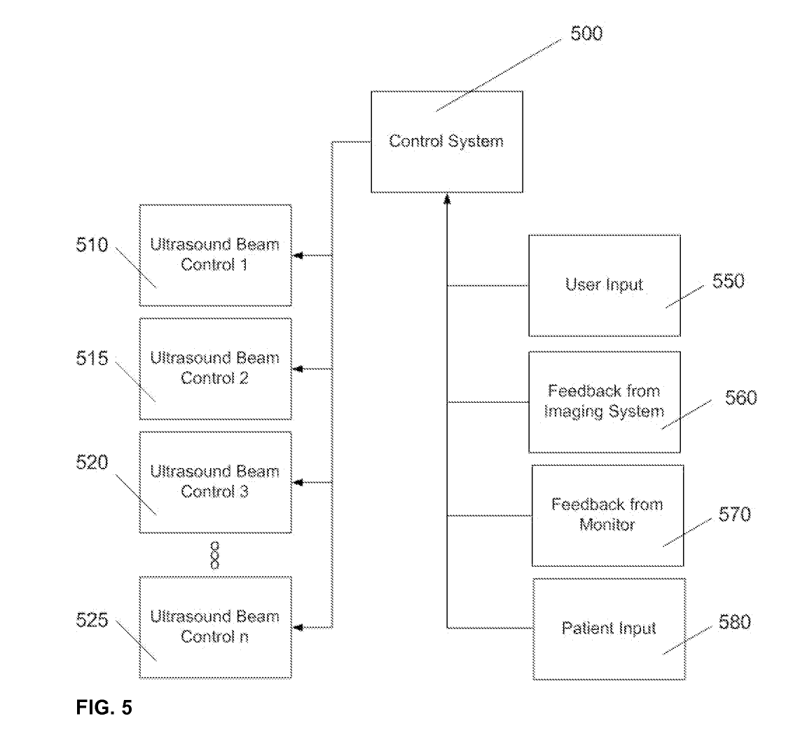

FIG. 5 shows a control block diagram. The frequencies, firing patterns, and intensities for the ultrasonic transducers 510, 515, 520, 525 (and, as applicable, additional ultrasound transducers as indicated by the ellipsis between ultrasound transducers 520 and 525) are controlled by control system 500 with control input from user by user input 550 and/or from feedback from imaging system 560 (either automatically or display to the user with actual control through user input 550), and/or feedback from a functional monitor (one or more of motion, thermal, etc.) 570, and/or the patient 580. If positioning of the ultrasound transducers is included as a control element, then control system 500 will control positioning as well.

The invention can be applied to a number of conditions including, but not limited to, addiction, Alzheimer's Disease, Anorgasmia, Attention Deficit Hyperactivity Disorder, Huntington's Chorea, Impulse Control Disorder, autism, OCD, Social Anxiety Disorder, Parkinson's Disease, Post-Traumatic Stress Disorder, depression, bipolar disorder, pain, insomnia, spinal cord injuries, neuromuscular disorders, tinnitus, panic disorder, Tourette's Syndrome, amelioration of brain cancers, dystonia, obesity, stuttering, ticks, head trauma, stroke, and epilepsy. In addition it can be applied to cognitive enhancement, hedonic stimulation, enhancement of neural plasticity, improvement in wakefulness, brain mapping, diagnostic applications, and other research functions. In addition to stimulation or depression of individual targets, the invention can be used to globally depress neural activity that can have benefits, for example, in the early treatment of head trauma or other insults to the brain.

All of the embodiments above, except those explicitly restricted in configuration to hit a single target, are capable of and usually would be used for targeting multiple targets either simultaneously or sequentially. The invention provides for hitting one or a plurality of targets in a single circuit or a plurality of neural circuits. Hitting multiple targets in a neural circuit in a treatment session is an important component of fostering a durable effect through Long-Term Potentiation (LTP) and/or Long-Term Depression (LTD) or enhances acute effects (e.g., such as treatment of post-surgical pain). In addition, this approach can decrease the number of treatment sessions required for a demonstrated effect and to sustain a long-term effect. Follow-up tune-up sessions at one or more later times may be required. In some cases, the neural structures will be targeted bilaterally (e.g., both the right and the left Insula) and in some cases unilaterally (e.g., the right Insula in the case of addiction).

The invention allows stimulation adjustments in variables such as, but not limited to, intensity, timing, firing pattern, and frequency, and position to be adjusted so that if a target is in two neuronal circuits the output of the transducer or transducers can be adjusted to get the desired effect and avoid side effects. Position can be adjusted as well. The side effects could occur because for one indication the given target should be up regulated and for the other down regulated. An example is where a target or a nearby target would be down regulated for one indication such as pain, but up-regulated for another indication such as depression.

The invention also covers contradictory effects in cases where a target is common to both two neural circuits in another way. This is accomplished by treating (either simultaneously or sequentially, as applicable) other neural-structure targets in the neural circuits in which the given target is a member to counterbalance contradictory side effects. This also applies to situations where a tissue volume of neuromodulation encompasses a plurality of targets. Again, an example is where a target or a nearby target would be down regulated for one indication such as pain, but up-regulated for another indication such as depression. This scenario applies to the Dorsal Anterior Cingulate Gyms (DACG). To counterbalance the down-regulation of the DACG during treatment for pain that negatively impacts the treatment for depression, one would up-regulate the Nucleus Accumbens or Hippocampus that are other targets in the depression neural circuit. A plurality of such applicable targets could be stimulated as well. One set of applied patterns can be applied to a given neural circuit to provide treatment for one condition and an alternative set of applied patterns is applied to the given neural circuit to provide treatment for another condition.

Another applicable scenario is the Nucleus Accumbens that is down regulated to treat addiction, but up regulated to treat depression. To counteract the down-regulation of the Nucleus Accumbens to treat depression but will negatively impact the treatment of depression that would like the Nucleus Accumbens to be up regulated, one would up-regulate the Caudate Nucleus as well. Not only can potential positive impacts be negated, one wants to avoid side effects such as treating depression, but also causing pain. These principles of the invention are applicable whether ultrasound is used alone, in combination with other modalities, or with one or more other modalities of treatment without ultrasound. Any modality involved in a given treatment can have its stimulation characteristics adjusted in concert with the other involved modalities to avoid side effects.

The various embodiments described above are provided by way of illustration only and should not be construed to limit the invention. Based on the above discussion and illustrations, those skilled in the art will readily recognize that various modifications and changes may be made to the present invention without strictly following the exemplary embodiments and applications illustrated and described herein. Such modifications and changes do not depart from the true spirit and scope of the present invention.

What is claimed is:

1. A method for ultrasound neuromodulation of one or a plurality of deep-brain targets comprising:
a. Providing one or a plurality of ultrasound transducers;
b. Aiming the beams of said ultrasound transducers at one or a plurality of applicable neural targets;
c. modulating the ultrasound transducers with patterned stimulation, whereby the one or a plurality of neural targets are each neuromodulated producing regulation selected from the group consisting of up-regulation and down-regulation.

2. The method of claim 1, wherein the variation is of one or a plurality selected from the group consisting of inter-pulse intervals and inter-train intervals.

3. The method of claim 1, wherein the pulse-burst trains are selected from the group consisting of fixed and varied.

4. The method of claim 1, wherein the inter-pulse-train intervals are selected from the group consisting of fixed and varied.

5. The method of claim 1, wherein the applied intensity pattern is selected from the group consisting of fixed and varied.

6. The method of claim 1, wherein the pattern applied is selected from the group consisting of random, theta-burst stimulation.

7. The method of claim 1 wherein the control system used for control of the patterns is selected from one or a plurality of inputs selected from the group consisting of user input, feedback from imaging system, feedback from functional monitor, and patient input.

8. The method of claim 1, wherein the relationship among applied frequency pattern, applied timing pattern, and applied intensity pattern is selected from the group consisting of independently varied, dependently varied, independently fixed, and dependently fixed.

9. The method of claim 1, wherein the pattern is varied during the course of neuromodulation.

10. The method of claim 1, wherein the effect of patterned ultrasonic neuromodulation is selected from one or more of the group consisting of acute effect, Long-Term Potentiation and Long-Term Depression.

11. The method of claim 1, wherein the applied pattern is selected from the group of synchronous with all ultrasound transducers using the same pattern and asynchronous with not all ultrasound transducers using the same pattern.

12. The method of claim 1, wherein the locations of the targets are selected from the group consisting of in the same neural circuit and in different neural circuits.

13. The method of claim 1, wherein the use of multiple ultrasound transducers is selected from one or a plurality of the group consisting of neuromodulation of the same target and neuromodulation of different targets.

14. The method of claim 1, wherein the pattern applied in used to avoid side effects elicited by neuromodulation of one or a plurality of structures selected from the group consisting of unintended structures and structures that need to be protected from neuromodulation.

15. The method of claim 1, wherein the applied pattern is selected from the group of where all targets receive the same pattern and all targets do not receive the same pattern.

16. The method of claim 1, wherein one set of applied patterns applied to a given neural circuit to provide treatment for one condition and an alternative set of applied patterns is applied to that neural circuit to provide treatment for another condition.

17. The method of claim 9, where one treated condition is the manic phase of bipolar disorder and the other treated condition is the depressive phase of bipolar disorder.

18. The method of claim 10, wherein the manic phase is treated with neuromodulation causing down-regulation and the depressive phase is treated with neuromodulation causing up-regulation.
